(19) United States
(12) Patent Application Publication
(10) Pub. No.: US 2014/0275603 A1
(43) Pub. Date: Sep. 18, 2014

(54) PROCESS FOR MAKING 2-NITRO-1-ETHANOL DERIVATIVES

(71) Applicants: Raymond J. Swedo, Mount Prospect, IL (US); George David Green, Cary, IL (US); Charles E. Coburn, Vernon Hills, IL (US)

(72) Inventors: Raymond J. Swedo, Mount Prospect, IL (US); George David Green, Cary, IL (US); Charles E. Coburn, Vernon Hills, IL (US)

(73) Assignee: ANGUS Chemical Company, Buffalo Grove, IL (US)

(21) Appl. No.: 14/000,502

(22) PCT Filed: Mar. 26, 2012
(86) PCT No.: PCT/US2012/030502

§ 371 Date: Sep. 18, 2013

Related U.S. Application Data
(60) Provisional application No. 61/472,747, filed on Apr. 7, 2011.

(51) Int. Cl. C07C 201/12; C07C 211/15; C07C 215/76; C07C 211/48; C07C 211/52; C07C 205/54; C07C 205/51

(52) U.S. Cl. CPC C07C 201/12; C07C 205/54; C07C 211/15; C07C 205/51; C07C 211/48; C07C 211/52; C07C 215/76
USPC 560/20; 568/704; 564/463; 560/74; 560/266; 564/305; 564/441; 564/443

(57) ABSTRACT

A process for making a 2-nitro-1-ethanol derivative of formula III: wherein R3 is as described herein is provided. Novel 2-nitro-1-ethanol derivatives provided.

[chemical structure]

### CROSS-REFERENCE TO RELATED APPLICATIONS

This application claims priority from provisional application Ser. No. 61/472,747, filed Apr. 7, 2011, which is incorporated herein by reference in its entirety.

### BACKGROUND

This invention relates generally to a process for making 2-nitro-1-ethanol derivatives. The invention also relates to novel compounds.

The compound 2-nitro-1-ethanol (2-NE) is an attractive synthesis intermediate because of its multiple functionality. For instance, reaction of the —OH group can yield esters, while the carbon alpha to the nitro group can participate in Michael reactions, Mannich reactions, and the like. In addition, the nitro group can be reduced to an amine.

In spite of its potential utility, 2-NE is not widely used because of problems associated with its synthesis. For instance, in Noland, Organic Syntheses, Collective Volume 5, John Wiley and Sons, New York, 1973, p. 833, a synthesis of 2-NE on a lab scale is described that uses an approximately 10 fold molar excess of nitromethane. Nitromethane is a detonable liquid and its handling therefore presents challenges, particular when used in large excesses.

In addition to the concerns associated with nitromethane, the 2-NE itself may begin to decompose during distillation, thus further hampering the synthesis. As a result of these problems, 2-NE is not easily prepared and therefore not readily available.

The problem addressed by this invention is the provision of 2-NE derivatives in a manner that avoids one or more of the problems and hazards associated with the prior art.

### STATEMENT OF INVENTION

We have now found that 2-NE derivatives may be readily prepared through a process that does not require the synthesis and isolation of the 2-NE compound as a precursor. Advantageously, therefore, the hazards associated with conventional processes may be mitigated or avoided.

In one aspect, there is provided a process for making 2-nitroethanol derivatives. The process comprises:

(a) providing a compound of formula I:

[chemical structure]

wherein R is H or CH₂OH, and R₁and R₂ are independently H, C₁-C₆ alkyl, halo substituted C₁-C₆ alkyl, aryl, or furanyl;

(b) converting the compound of formula Ito a compound of formula II:

[chemical structure]

wherein R₃ is the residue of an alpha carbon reactant or R₃ is —CH₂-R₄ wherein R₄ is the residue of an alcohol group reactant;

(c) converting the compound of formula II to a 2-nitroethanol derivative of formula III:

[chemical structure]

In another aspect, the invention provides compounds of the formula III-1:

[chemical structure]

wherein R₅ is C₁-C₆ alkyl, C₃-C₈ cycloalkyl, aryl, or aralkyl-, wherein cycloalkyl and aryl are optionally substituted with 1 or 2 of C₁-C₆ alkyl, nitro, halo, alkoxy, or carbonyl.

In a further aspect, the invention provides compounds of the formula III-2:

[chemical structure]

wherein R₆ and R₇ are independently H, C₁-C₆ alkyl, C₃-C₈ cycloalkyl, aryl, or aralkyl-, wherein cycloalkyl and aryl are optionally substituted with 1 or 2 of C₁-C₆ alkyl, nitro, halo, alkoxy, or carbonyl.

In still another aspect, the invention provides compounds of the formula III-3:

[chemical structure]

wherein R₈ is C₁-C₆ alkyl, C₃-C₈ cycloalkyl, aryl, or aralkyl-, wherein cycloalkyl and aryl are optionally substituted with 1 or 2 of C₁-C₆ alkyl, nitro, halo, alkoxy, or carbonyl.

In yet another aspect, the invention provides compounds of the formula III-4:

[chemical structure]

wherein R₉, R₁₀, R₁₁, and R₁₂ are independently CN, CO₂H, CO₂R₁₃, COR₁₃, H, C₁-C₆ alkyl, C₃-C₈ cycloalkyl, aryl, or aralkyl-, with the proviso that at least one of R₉, R₁₀, R₁₁, and R₁₂ is CN, CO₂H, CO₂R₁₃, or COR₁₃; and wherein R₁₃ is C₁-C₆ alkyl, C₃-C₈ cycloalkyl, aryl, or aralkyl-, and wherein cycloalkyl and aryl are optionally substituted with 1 or 2 of C₁-C₆ alkyl, nitro, halo, alkoxy, or carbonyl.

### DETAILED DESCRIPTION

Numeric ranges are inclusive of the numbers defining the range. Unless otherwise indicated, ratios, percentages, parts, and the like are by weight.

“Alkyl” as used in this specification encompasses straight and branched chain aliphatic groups having the indicated number of carbon atoms. If no number is indicated, then 1-10, alternatively 1-6, alkyl carbons are contemplated. Preferred alkyl groups include, without limitation, methyl, ethyl, propyl, isopropyl, butyl, isobutyl, sec-butyl, tert-butyl, pentyl, and hexyl.

The term “cycloalkyl” refers to saturated and partially unsaturated cyclic hydrocarbon groups having the indicated number of ring carbon atoms. Fully saturated groups are preferred. Preferred cycloalkyl include, without limitation, cyclopropyl, cyclobutyl, cyclopentyl, cyclohexyl, cycloheptyl, and cyclooctyl. “Cyclic diether” refers to a cycloalkyl in which two of the ring carbon atoms are replaced with oxygen atoms.

An “aryl” group is a C6-C12 aromatic moiety comprising one to three aromatic rings. Preferably, the aryl group is a C6-C10 aryl group. Preferred aryl include, without limitation, phenyl, naphthyl, anthracenyl, and fluorenyl. More preferred are phenyl and naphthyl.

The term “aralkyl-” refers to aryl-C₁-C₆ alkyl-. A preferred aralkyl group is benzyl.

“Halo” refers to chloride, bromide, fluoride, or iodide. Chloride and bromide are preferred. Chloride is more preferred.

The term “blocking group precursor” refers to a reagent that reacts with tris(hydroxymethyl)nitromethane to form the compound of formula I. Examples of suitable blocking group precursors include, for instance: geminal diethers such as 2,2-dialkoxypropanes and 2,2-dimethoxypropane; aldehydes such as formaldehyde, propionaldehyde, butyraldehyde, benzaldehyde, furfural, chloroacetaldehyde; and ketones such as 2-propanone or butanones (e.g., 2 or 3 butanone), cyclopentanone, cyclohexanone, acetophenone, propiophenone, and benzophenone. Preferred are geminal diethers and ketones.

The formula I compound contains an acidic hydrogen at the carbon alpha to the nitro group or it contains a hydroxy-methyl group. Advantageously, the other otherwise reactive sites of the 2-nitroethanol molecule are blocked in the compound of formula I from further reaction by blocking groups. Consequently, the compound may undergo targeted reactions at the open sites without interference by the other now blocked functionalities.

Compounds of formula I in which R is CH₂OH may be prepared by reacting tris(hydroxymethyl)nitromethane with a blocking group precursor. The reaction can be readily carried out by those skilled in the art. Examples of suitable procedures are described in U.S. Pat. Nos. 2,297,921 and 2,368,071, each of which is incorporated by reference herein in its entirety. Typically, the reaction between the blocking group precursor, such as aldehyde or ketone, and the tris(hydroxymethyl)nitromethane may be carried out in the presence of a catalytic amount of an acid, such as concentrated hydrochloric acid, sulfuric acid, or methanesulfonic acid. In some embodiments, it may be preferable to use an excess of the blocking group precursor. It may also be desirable to conduct the reaction in the presence of a liquid that is capable of azeotropic removal of the produced water, such as pentane. Once the desired level of reaction has occurred, the product may be purified by techniques well known to those skilled in the art including, for instance, through neutralization of the acid catalyst followed by washing, drying, and distillation. Compounds of formula I in which R is H may be readily prepared simply by base catalyzed cleavage of the CH₂OH (R group) of the product from the foregoing reaction.

The tris(hydroxymethyl)nitromethane used in the above reaction is commercially available or it may be readily prepared, for instance through the reaction of nitromethane and formaldehyde. In some embodiments, in may be desirable to prepare the formula I compound through a one pot synthesis starting from nitromethane. According to this embodiment, the nitromethane is first reacted with the formaldehyde, followed by reaction with the blocking group precursor to yield the formula I compound. As a one pot synthesis, isolation and/or purification of intermediate compounds is advantageously not required.

When the formula I compound contains a hydrogen at the alpha carbon (i.e., R is H), the compound may undergo carbon-carbon bond forming reactions at this site to yield useful compounds or their precursors. The group added to the alpha-carbon is referred to herein as the “residue of an alpha carbon reactant.” Examples of reactions providing such residue include, for instance, Michael reaction, Henry reaction, and Mannich reaction.

The Michael reaction is a well known and highly useful method for the formation of C—C bonds. In the invention, the formula I compound (wherein R is H) functions as a Michael donor. A wide variety of compounds may function as the Michael acceptor including, but not limited to, acrylonitrile, acrylic acid, methacrylic acid, acrylic acid esters, methacrylic acid esters, acrylamides, methacrylamides, α,β-unsaturated aldehydes, ketones, and esters. The product of the Michael reaction is a compound of formula II in which R³ is the residue of the Michael acceptor.

The Mannich reaction is also a well known and highly useful bond forming method that involves use of an amine and an aldehyde as co-reactants. In the invention, the formula I compound (wherein R is H) functions as the nucleophile in the reaction. A wide variety of reagents may function as the amine and the aldehyde including, for instance, ammonia, methylamine, dimethylamine, the propylamines and diamines, the butylamines and diamines, the pentylamines and diamines, the hexylamines and diamines; mixed alkylamines such as methylethylamine; cyclic amines such as cyclopentylamine and cyclohexylamine; heterocyclic amines such as piperadine and piperazine; aromatic amines such as aniline and substituted anilines; formaldehyde, dimethoxypropane, paraformaldehyde, acetaldehyde, propionaldehyde, butyraldehyde, isobutyraldehyde, benzaldehyde. The product of the Mannich reaction is a compound of formula II in which R³ is the residue of the amine and aldehyde co-reactants.

When the formula I compound contains a CH₂OH at the R position, the OH may react with a variety of reagents. The product of the reaction between the hydroxy and the reagent is referred to in this specification as the “residue of an alcohol group reactant.” Examples of suitable OH reactions include ester formation and Mannich reactions.

Once the desired compound of formula II is formed, the blocking group is removed from the molecule, thus yielding the formula III compound. The blocking group may be removed using methods familiar to those skilled in the art, such as described by J. B. Morin and J. K. Sello, Organic Letters, 12 (15), 3522 (2010), or in EP 0348223 A2. Typically, the removal may be carried out, for instance, by acid catalyzed cleavage of the CR₁R₂ moiety followed by base catalyzed cleavage of the CH₂OH residues.

In some embodiments, compounds of formula III are of the formula III-I:

[chemical structure]

wherein R₅ is C₁-C₆ alkyl, C₃-C₈ cycloalkyl, aryl, or aralkyl-, wherein cycloalkyl and aryl are optionally substituted with 1 or 2 of C₁-C₆ alkyl, nitro, halo, alkoxy, or carbonyl. Preferred compounds of formula III-1 include compounds wherein R₅ is H. Also preferred are compounds wherein R₅ is C₁-C₃ alkyl, more preferably methyl.

In some embodiments, compounds of formula III are of the formula III-2:

[chemical structure]

wherein R₆ and R₇ are independently H, C₁-C₆ alkyl, C₃-C₈ cycloalkyl, aryl, or aralkyl-, wherein cycloalkyl and aryl are optionally substituted with 1 or 2 of C₁-C₆ alkyl, nitro, halo, alkoxy, or carbonyl. Preferred compounds of formula III-2 include compounds wherein R₆ and R₇ are independently H or C₁-C₆ alkyl. Also preferred are compounds wherein one of R₆ and R₇ is H and the other is C₁-C₆ alkyl. Further preferred are compounds wherein R₆ and R₇ are both independently selected C₁-C₆ alkyl groups.

In some embodiments, compounds of the formula III are of the formula III-3:

[chemical structure]

wherein R₈ is C₁-C₆ alkyl, C₃-C₈ cycloalkyl, aryl, or aralkyl-, wherein cycloalkyl and aryl are optionally substituted with 1 or 2 of C₁-C₆ alkyl, nitro, halo, alkoxy, or carbonyl. Preferred compounds of formula III-3 include those wherein R₈ is C1-C6 alkyl.

In some embodiments, compounds of the formula III are of the formula III-4:

[chemical structure]

wherein R₉, R₁₀, R₁₁, and R₁₂ are independently CN, CO₂H, CO₂R₁₃, COR₁₃, H, C₁-C₆ alkyl, C₃-C₈ cycloalkyl, aryl, or aralkyl-, with the proviso that at least one of R₉, R₁₀, R₁₁, and R₁₂ is CN, CO₂H, CO₂R₁₃, or COR₁₃; and wherein R₁₃ is C₁-C₆ alkyl, C₃-C₈ cycloalkyl, aryl, or aralkyl-, and wherein cycloalkyl and aryl are optionally substituted with 1 or 2 of C₁-C₆ alkyl, nitro, halo, alkoxy, or carbonyl. Preferred are compounds wherein R₁₁ and R₁₂ are H and R₉ and R₁₀ are independently H or CN, CO₂H, CO₂R₁₃, or COR₁₃. Also preferred are compounds wherein R₁₁ R₁₂, and R₉ are H and R₁₀ is CN, CO₂H, CO₂R₁₃, or COR₁₃.

The compounds of formula III, III-1, III-2, III-3, and III-4 find utility in a variety of applications. For instance, such compounds may be used as intermediates in the synthesis of commercial chemicals or pharmaceutical agents.

Some embodiments of the invention will now be described in detail in the following Examples.

### EXAMPLES

### Example 1

5-Nitro-2,2-dimethyl-5-hydroxymethyl-1,3-dioxane is made from 0.33 moles of tris-(hydroxymethyl)-nitromethane (TN, TRIS-NITRO®, ANGUS Chemical Company) and 3.4 moles of acetone in refluxing pentane containing 0.1 mL of methanesulfonic acid. Water was removed as the pentane azeotrope. When the theoretical amount of water has been removed, the pentane and excess acetone are removed by rotary evaporation at a bath temperature of ≦35° C. at reduced pressure. The resulting crude product is taken up in 750 mL of ethyl acetate. The resulting solution is washed twice with 125 mL portions of saturated aqueous sodium bicarbonate solution, and then it is dried over anhydrous magnesium sulfate. The drying agent is removed by filtration, and the solvent removed by rotary evaporation to give 31.5 grams (50% yield) of the blocked TN.

### Example 2

5-Nitro-2,2-dimethyl-1,3-dioxane is made by mixing 5-nitro-2,2-dimethyl-5-hydroxymethyl-1,3-dioxane (0.03 moles) with about 70 mL of 10 wt. % sodium hydroxide solution, and the solution is stirred at 60° C. for about 60 minutes. The solution is cooled to about 5° C., and is acidified to pH 5 with concentrated acetic acid. The precipitated solid is filtered off and dried to give a 92% yield of product.

### Example 3

3-(2,2-Dimethyl-5-nitro-1,3-dioxan-5-yl)propanenitrile is made by slowly adding 1 mole of 2,3,4,6,7,8,9,10-octahydropyrimidol[1,2-α] (DBU) to a solution of 1 mole of 5-nitro-2,2-dimethyl-1,3-dioxane and 1 mole of acrylonitrile in 2 L of acetonitrile. The temperature of the reaction mixture is kept at <20° C. during the addition. The solution is then stirred at room temperature for about 7 hours. The solvent is removed by rotary evaporation to give a crude product which is taken up in ethyl acetate. The ethyl acetate solution is washed with 6N hydrochloric acid solution to remove the DBU. The solution is then washed with saturated aqueous sodium bicarbonate solution, and is dried over anhydrous magnesium sulfate. The solvent is removed by rotary evaporation to give the product in about 75% yield.

### Example 4

5-Hydroxy-4-(hydroxymethyl)-4-nitropentanenitrile is made by heating a solution of 3-(2,2-Dimethyl-5-nitro-1,3-dioxan-5-yl)propanenitrile and 75 mL of concentrated hydrochloric acid in 2.5 L of methanol at 35 -40° C. for about 1 hour. The bulk of the methanol is removed by rotary evaporation at a bath temperature of <40° C. The resulting residue is mixed with about 500 mL of water, and the pH is adjusted to about 6-7 by adding dilute aqueous sodium hydroxide solution. The solution is then extracted with several portions of ethyl ether. The ether solution is washed with saturated aqueous sodium chloride, and is then dried over anhydrous magnesium sulfate. The solvent is removed by rotary evaporation to give the product in about 90% yield.

### Example 5

4-Nitrobutanenitrile is made by heating a solution of 1 mole of 5-hydroxy-4-(hydroxymethyl)-4-nitropentanenitrile in 2 L of 10% aqueous sodium hydroxide solution at 60° C. for about 1 hour. The solution is then cooled to about 5° C., and then it is acidified to pH 5 by the addition of glacial acetic acid. The resulting mixture is extracted with several portions of ethyl ether. The ether solution is washed with water and is dried over anhydrous magnesium sulfate. The solvent is removed by rotary evaporation to give the product in about 90% yield.

1. A process for making a 2-nitroethanol derivative, the process comprising:
(a) providing a compound of formula I: [chemical structure]
wherein R is H or CH₂OH, and R₁and R₂ are independently H, C₁-C₆ alkyl, halo substituted C₁-C₆ alkyl, aryl, or furanyl;
(b) converting the compound of formula I to a compound of formula II: [chemical structure]
wherein R₃ is the residue of an alpha carbon reactant or R₃ is —CH₂-R₄ wherein R₄ is the residue of an alcohol group reactant;
(c) converting the compound of formula II to a 2-nitroethanol derivative of formula III:

[chemical structure]

2. The process of claim 1 wherein R is CH₂OH and the compound of formula I is prepared by reacting tris(hydroxymethyl)nitromethane with a blocking group precursor.

3. The process of claim 1 wherein R is H and the compound of formula I is prepared by reacting tris(hydroxymethyl)nitromethane with a blocking group precursor followed by treatment with base to remove a —CH₂OH group.

4. The process of claim 2 wherein the blocking group precursor is a geminal diether compound, an aldehyde compound, or a ketone compound.

5. A compound of formula III-1: [chemical structure]
wherein R₅ is C₁-C₆ alkyl, C₃-C₈ cycloalkyl, aryl, or aralkyl-, wherein cycloalkyl and aryl are optionally substituted with 1 or 2 of C₁-C₆ alkyl, nitro, halo, alkoxy, or carbonyl.

6. A compound of formula III-2: [chemical structure]
wherein R₆ and R₇ are independently H, C₁-C₆ alkyl, C₃-C₈ cycloalkyl, aryl, or aralkyl-, wherein cycloalkyl and aryl are optionally substituted with 1 or 2 of C₁-C₆ alkyl, nitro, halo, alkoxy, or carbonyl.

7-8. (canceled)
